(19) United States
(12) Patent Application Publication
(10) Pub. No.: US 2019/0209828 A1
(43) Pub. Date: Jul. 11, 2019

(54) IONTOPHORETIC DEVICE COMPRISING A MOVEMENT SENSOR

(71) Applicant: L'OREAL, Paris (FR)

(72) Inventor: Thi Hong Lien PLANARD-LUONG, Chevilly La Rue (FR)

(73) Assignee: L'OREAL, Paris (FR)

(21) Appl. No.: 16/333,775

(22) PCT Filed: Sep. 19, 2017
(86) PCT No.: PCT/EP2017/073676

§ 371 Date: Mar. 15, 2019

(30) Foreign Application Priority Data
Sep. 20, 2016 (FR) 1658821

(51) Int. Cl. A61N 1/04; A61B 5/053; A61N 1/32; A61N 1/36

(52) U.S. Cl. CPC A61N 1/0448; A61B 5/0531; A61N 1/36017; A61N 1/327; A61N 1/328; A61N 1/325

(57) ABSTRACT

The invention relates to a device (1) for detecting a movement of a cosmetic end piece connected to an electrical generator (10) and comprising an electrode (2) intended to be moved over the skin (100), a counterelectrode (3) and a microcontroller (11) connected to the electrical generator (10) and capable of applying a controlled electrical input signal Uᵢₙ(t) to the electrode (2), the device comprising a sensor (4) that is capable of measuring a detected electrical signal U_{det}(t) between the electrode (3) and the skin and of sending the detected electrical signal U_{det}(t) to a subtracter (5) which measures any distortion 5(t) between the detected electrical signal U_{det}(t) and a reference electrical signal U_{ref} and provides, as an output, a zero exploitation signal to the electrode (2) if the measured distortion 5(t) is greater than a predetermined threshold voltage Uₒ.

The present invention relates to devices for performing a cosmetic treatment of keratin materials.

More generally, a cosmetic product is a product as defined in Regulation (EC) No. 1223/2009 of the European Parliament and Council of 30 Nov. 2009 relating to cosmetic products.

The invention relates in particular to devices for treating keratin materials with the aid of an electric current.

The expression “human keratin materials” is understood as meaning mainly the skin, notably of the body or of the face, or the scalp, the nails or the hair.

### TECHNOLOGICAL BACKGROUND

It is known that the application of an electric current to the skin can promote the penetration of an active agent. It is thus known to treat human keratin materials with the aid of iontophoretic devices. Iontophoresis allows the diffusion of active agents through the skin by virtue of electrical stimulation in a non-invasive manner. The current administered can be adjustable in terms of intensity and polarity (anodic or cathodic current). The transcutaneous diffusion of molecules via iontophoresis is based on two principles, namely electrorepulsion and electroosmosis.

The radio frequency is based on the emission of very high frequency electromagnetic waves passing through the skin, which produce heat in the subcutaneous tissues. The heat produced acts deep down, without burning the surface, i.e. the skin. It is possible to heat up to 55° C. deep down, which stimulates the fibroblasts, which manufacture new elastin and collagen fibres. In parallel, the heat induces shrinkage of the pre-existing collagen fibres.

This revolutionary technique treats skin deep down, and stimulates the production of new collagen, which improves the firmness and tautness of the skin.

Electroporation (also known as needle-free mesotherapy) is a method which induces opening of the skin's water channels (electropores) via the effect of specific waveforms. Better transport of the active principles of the cosmetics used is obtained. Various techniques exist, depending on the waveform and the application methods used. Electroporation has a shorter duration of application than conventional iontophoresis, it is entirely painless and allows controlled transport of large amounts of active principles.

The use of electrostimulation in aesthetics makes it possible to firm and tonify certain slackened muscles of the face and body. The particular feature of nerve and muscle cells is that they are excitable. This property allows them to respond to an electrical “stimulus”: when this stimulus is initiated on a nerve cell, it propagates very quickly along the membrane thereof. After having crossed the “neuromuscular junction”, it travels across the membrane of the muscle cells, and the excitation is then transmitted deep down and triggers contraction.

Several types of appliances for treating the skin with microcurrents which supply electrical pulses of different forms and frequencies have already been proposed.

U.S. Pat. No. 8,731,657 describes a device comprising a microcontroller configured to generate a plurality of high and low frequency electromagnetic waveforms of different amplitudes.

U.S. Pat. No. 6,684,107 describes a wireless device comprising a generator for periodically generating a microcurrent with a frequency of less than 16 Hz.

It has already been proposed to control the movement of the end piece of devices for treating the skin with an electric current.

For example, patent application US 2016/106505 discloses such a device comprising a system for controlling the speed of the end piece.

Patent application Ep 1 412 142 discloses an electric razor comprising a heat detector which stops the razor if the temperature is too high on contact with the skin.

There is a need to further improve devices for cosmetic treatment with the aid of an electric current which promote the penetration of active agents so as to increase the efficacy of the treatments.

There is a need to benefit from a device for treating keratin materials with the aid of an electric current, which exhibits good efficacy and can be used with comfort and in total safety, above all for dynamic treatment.

There is also a need to benefit from such a safe device, which limits the tingling, burning or heating of the treated zone.

There is also a need to have available such a device which is sparingly sensitive to parasites.

There is a need to perform a controlled dynamic treatment.

### DEFINITION OF THE INVENTION

According to one of its aspects, a subject of the invention is thus a device for detecting a movement of a cosmetic end piece connected to an electrical generator and comprising an electrode intended to be moved over the skin, a counterelectrode and a microcontroller connected to the electrical generator and capable of applying a controlled electrical input signal Uin(t) to the electrode, the device comprising a sensor that is capable of measuring a detected electrical signal Udet(t) between the electrode and the skin and of sending the detected electrical signal Udet(t) to a subtracter which measures any distortion δ(t) between the detected electrical signal Udet(t) and a reference electrical signal Uref and provides, as an output, a zero exploitation signal to the electrode if the measured distortion δ(t) is greater than a predetermined threshold voltage Uo.

By virtue of the particular feature of the invention which consists in measuring a signal distortion, the device according to the invention is particularly reliable.

With this device, the risk of burning and damaging the bodily surface treated with the end piece is greatly diminished.

Specifically, as soon as the end piece remains in contact with the skin in the same position for a prolonged period, the dielectric properties of the skin change. The electrical signal detected between the electrode and the counterelectrode, separated by the skin which has evolutive dielectric properties, changes as a function of time.

The device according to the invention is precise. In safe mode, the energy delivered is small to avoid any static problem. The user safety is markedly improved.

The invention also relates to an appliance for the cosmetic treatment of keratin materials with the aid of an electric current, comprising a device as described previously.

Sensor

The electrode and the skin (functioning as an electrode or counterelectrode) placed face-to-face constitute a capacitor whose capacitance is:

C=ε0*εr*A/d

with:

ε0=8.854 187 pF/m vacuum permittivity

εr relative permittivity of the material present between the two electrodes, for example the stratum corneum,

A: surface area facing between the electrode and the counterelectrode

d: distance between the electrode and the counterelectrode.

### First Case: Absence of an Object Between the Two Electrodes

ε r≈1(air)⇒C≈ε0 A/d

This first case arises, for example, when the end piece is far away from the skin.

### Second Case: Presence of an Object Between the Two Electrodes

⇒(εr≈4)

The counterelectrode may be in contact with the user's fingers, for example.

C=(ε 0.ε r.A)/d

When the mean εr becomes greater than 1 in the presence of the insulating object, C increases. Measurement of the increase in the value of C makes it possible to detect the presence of the insulating object.

### Third Case: Presence of a Conductive Object Between the Electrode and the Counterelectrode

C=(ε 0 ε r A)/d−e

with:

ε r≈1(air)⇒C≈ε 0(A/d−e)

e: distance between the electrode and the conductive object

The presence of a conductive object is also reflected by an increase in the value of C.

When the electrode is in contact with an area of the body, the permittivity of the skin located close to the electrode changes as a function of time. Specifically, the current transmitted by the electrode onto the skin changes the skin's physicochemical properties. This change brings about a variation of the permittivity as a function of time.

The sensor records the change in capacitance as a function of time. When the capacitance becomes greater than a predetermined value, the skin may deteriorate.

Power Supply System

The device comprises a power supply system for exposing the keratin materials to an electrical treatment current in a zone of application of the composition.

A “power supply system” is understood to be an electrical assembly that is able to induce a potential difference between one or more electrodes and at least one counterelectrode. If the application end piece is placed on the face and if the counterelectrode is held in one hand, the potential difference is established between the face and the hand.

Application End Piece

The application end piece may or may not be conductive.

It may comprise several application members, notably in the form of balls or spikes. The multiplicity of application members promotes good distribution of the composition in a continuous manner, and improves the ionization of the composition during the treatment, where appropriate. The multiplicity of application members also makes it possible to improve the massaging effect during treatment.

Advantageously, the application end piece comprises at least one ball that is movable about a centre of rotation.

As a variant, the application members may have any profile, notably a cylindrical profile, for example in the form of rollers, or a non-cylindrical profile, for example an ovoid or discoid, filamentous or triangular shape.

The application members may be made of plastic or metal.

The outer surface of the end piece may be totally inert from a chemical point of view with regard to the composition applied and the keratin materials. The outer surface may be covered with a varnish. The outer surface may be polished. The outer surface may comprise a biocidal material, where appropriate.

Electrodes

The electrode may be positively charged (anode) or negatively charged (cathode). This electrode may be located in the application end piece, so as to ensure the passage of the electric current to the skin. If the electrode is located inside the end piece, it does not come directly into contact with the keratin materials, and is referred to as a remote electrode.

An electrode may be in the form of a ball, spike, stud or tongue, for example. The device may comprise a single or several electrodes.

The electrode may be flat, for example in the form of a flat disc or of a polygon. The electrode may be hollow, being formed for example by stamping or bending an electrically conductive metal sheet. The electrode may be porous. The electrode may be formed by a surface treatment for making the first zone conductive.

A “counterelectrode” is understood to be an electrode that is charged with an opposite sign to the electrode: negative, positive or alternating or connected to the earth of the generator. In general, said counterelectrode is located on the body of the device or on a handpiece. The counterelectrode is intended to come into contact with an area of the body of the person undergoing the care. In one embodiment, the counterelectrode is located on the end piece. If this is the case, it is separated from the electrode by an insulating space.

If the electrode is housed inside the end piece, it may notably be spaced apart from the outer wall of the application end piece by a distance of between 0.2 mm and 5 mm. This distance is the shortest measurable distance between the electrode and the outer surface of the application end piece. It is measured between any points of the electrode and of the outer surface of the application end piece, as long as the distance measured is the shortest distance.

Electrical Parameters

The electrical power source may include any cell or any accumulator. The voltage between the terminals is, for example, between 1.2 V and 24 V, preferably between 1.2 and 3.3 V. Where appropriate, the passage of the current can create spot heating.

The peak voltage of the pulses may range up to 400 V, but the energy delivered does not exceed 80 mJ per pulse or the power of the pulses does not exceed 5 W.

Reference Electrical Signal

The signal at time t is compared with the signal at time t+Δt. When the applicator is moving over the skin, the relative distortion will be of high frequency, but when the applicator is stationary, the relative distortion is rather of low frequency or even non-existent. As the method is relative over the same area of skin, the difference between different types of skin is not a problem. The Δt remains very small.

Complementary Cosmetic Treatments

Advantageously, the device comprises a source of heat, of light, for setting the application end piece in motion, notably a sonic, ultrasonic, radio frequency or electroporation vibration.

Composition

The device according to the invention may comprise a reservoir containing a cosmetic composition. The reservoir may or may not be removable.

Preferably, the cosmetic composition has a viscosity, measured at 25° C. and at a normal atmospheric pressure of 1.013×10⁵ Pa, of less than 0.5 Pa·s, more preferably less than 0.3 Pa·s, better still less than 0.2 Pa·s, notably in the range from 0.1 to 0.2 Pa·s, better still from 0.04 to 0.175 Pa·s.

The viscosity of the composition is measured at 25° C. and at a normal atmospheric pressure of 1.013×10⁵ Pa using a Rheomat 180 machine (from the company Lamy), equipped with an MS-R1, MS-R2, MS-R3, MS-R4 or MS-R5 spindle chosen depending on the consistency of the composition, and rotating at a rotation speed of 200 rpm. The measurement is taken after 10 minutes of rotation. The viscosity measurements are taken at most 1 week after production.

In the context of the invention, the spindles MS-R3 or MS-R2 or, for the most fluid compositions, MS-R1 will be used.

It is possible to use at least one cosmetic or dermatological composition with the device.

The composition(s) used may be in any form, for example in the form of an aqueous solution, an oil, an emulsion, a powder or a gel. The composition(s) used may also be sprayed onto the skin.

When the composition(s) used are in the form of a powder or gel, the latter can take on the shape of the electrode to which it is applied, as mentioned above. One or more compositions may be applied. To this end, the reservoir may comprise several compartments.

The composition(s) may comprise an active agent.

Advantageously, the composition is chosen from:

a face care or body care composition, notably comprising in particular an active agent chosen from humectant or moisturizing active agents, anti-ageing active agents, for example, depigmenting active agents, active agents that act on cutaneous microcirculation or seboregulating, anti-acne, filling, vitamin active agents;
a temporary, semi-permanent or permanent composition for making up the face or body,

a hair composition, notably a composition for washing the hair, for hair care or conditioning, for temporary form retention or shaping of the hair, for the temporary, semi-permanent or permanent dyeing of the hair, or for relaxing or permanent-waving, notably a composition for relaxing, dyeing or bleaching the roots and hair,

a composition for the scalp, in particular an antidandruff composition, a composition for preventing hair loss or for promoting regrowth of the hair, an anti-seborrhoeic, anti-inflammatory, anti-irritation or soothing composition, a mark-preventing composition or a composition for stimulating or protecting the scalp.

The device may be used in various cosmetic or dermatological treatments, for example for combating wrinkles, herpes, acne or for redensifying the skin or the hair.

### Preferred Embodiments

Preferably, the device according to the invention has one or more of the following features, on their own or in combination:
a filter is connected to the sensor output, notably a low-pass filter;
it comprises a phase shifter upstream of the sensor;
the distortion δ(t) is exploited by an operational amplifier mounted as a current integrator;
the microcontroller generates a controlled electrical signal cut up by a clock signal and the sensor performs detection that is synchronous with the clock signal.

Preferably, the appliance according to the invention has one or more of the following features, on their own or in combination:
it comprises a source of heat, of light, for setting the application end piece in motion, notably a sonic, ultrasonic, radio frequency or electroporation or iontophoretic or electrostimulatory vibration;
it comprises a cosmetic product reservoir;
it is an electrostimulation device.

### DESCRIPTION OF THE FIGURES

The invention will be better understood on reading the following detailed description of non-limiting exemplary embodiments of the invention and on examining the appended, schematic and partial drawing, in which:

FIG. 1 shows the general structure of a device according to the invention,

FIG. 2 shows two signals U(t):
a signal emitted on the electrode by the microcontroller, and
a signal detected by the sensitive sensor.

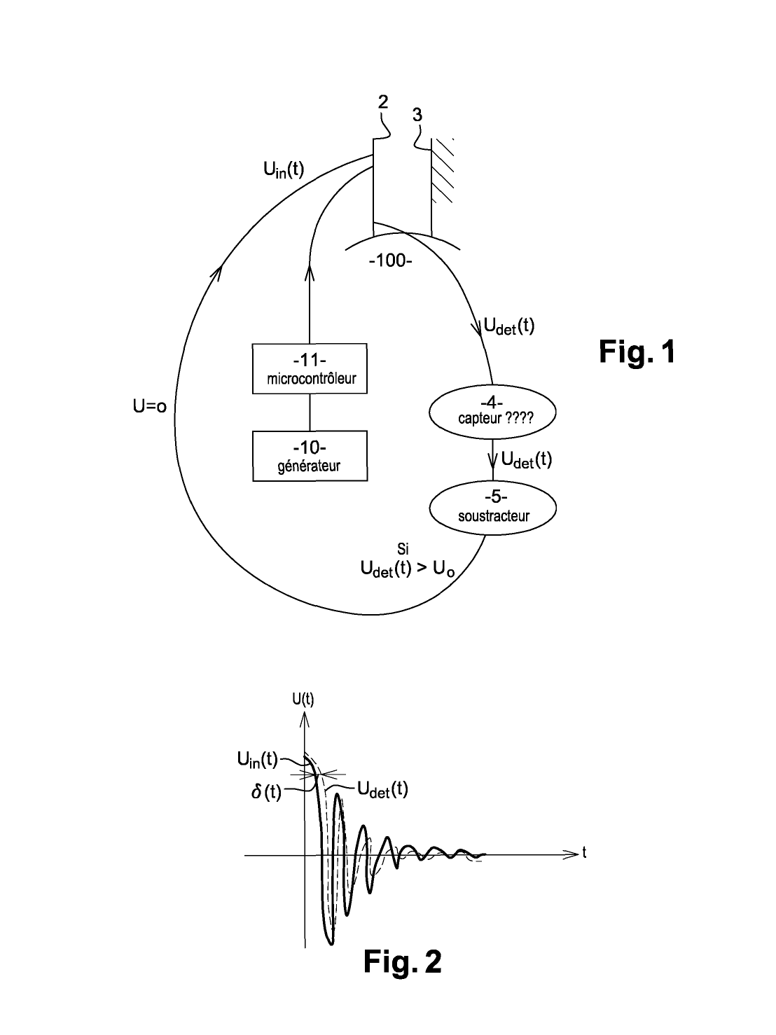

In FIG. 1, the device 1 comprises an electrode 2 and a counterelectrode 3. The electrode 2 receives an electrical input signal Uᵢₙ(t) from a generator 10 connected to a microcontroller 11. An electrical signal U_{det}(t) between the electrode 2 and the skin is detected by a sensor 4 and sent to a subtracter 5 which measures any signal distortion δ(t). It provides, as an output, a zero exploitation signal Uₑₓₚ(t) to the electrode 4 if the measured distortion δ(t) is greater than a predetermined threshold U₀.

In principle, the sensors 4 work with an RC oscillating circuit. A minimal variation in capacitance suffices to influence its oscillation amplitude. The evaluation electronics convert it into a switching signal. The user can adjust the sensitivity via a potentiometer.

FIG. 2 shows an input signal Uᵢₙ(t) in dampened sinusoidal form. The detected signal U_{det}(t) shows a phase shift relative to the input signal. This phase shift is compared with a threshold voltage value U₀. This comparison determines the signal that is sent to the electrode. This signal is zero if the phase shift is greater than the threshold voltage. It remains unchanged if the phase shift is less than or equal to the threshold voltage.

The invention is not limited to the examples illustrated. In particular, the features of the various exemplary embodiments which have just been described can be combined with each other.

1. Device for detecting a movement of a cosmetic end piece connected to an electrical generator and comprising an electrode intended to be moved over the skin, a counterelectrode and a microcontroller connected to the electrical generator and capable of applying a controlled electrical input signal Un(t) to the electrode, wherein the device comprises a sensor that is capable of measuring a detected electrical signal Udet(t) between the electrode and the skin and of sending the detected electrical signal Udet(t) to a subtracter which measures any distortion 8(t) between the detected electrical signal Udet(t) and a reference electrical signal Uref and provides, as an output, a zero exploitation signal to the electrode if the measured distortion 8(t) is greater than a predetermined threshold voltage Uₒ.

2. The device according to claim 1, wherein a filter is connected to the output of the sensor, notably a low-pass filter.

3. The device according to claim 1, wherein it comprises a phase shifter upstream of the sensor.

4. The device according to claim 1, wherein it comprises an operational amplifier mounted as a current integrator which exploits the distortion 8(t).

5. The device according to claim 1, wherein the microcontroller generates a controlled electrical signal and a clock signal cuts up the controlled electrical signal, and in that the sensor performs detection that is synchronous with the clock signal.

6. Appliance for the cosmetic treatment of keratin materials with the aid of an electric current, wherein it comprises a device according to claim 1.

7. The appliance according to claim 6, wherein it comprises a source of heat, of light, for setting the application end piece in motion, notably a sonic, ultrasonic, radiofrequency or electroporation or iontophoretic or electrostimulatory vibration.

8. The appliance according to claim 6, wherein it comprises a cosmetic product reservoir.

9. The appliance according to claim 6, wherein it is an electrostimulatory device.
